(19) United States
(12) Patent Application Publication
(10) Pub. No.: US 2024/0093068 A1
(43) Pub. Date: Mar. 21, 2024

(54) ADHESIVE WITH THERMALLY REVERSIBLE, COVALENT CROSSLINKS

(71) Applicant: 3M INNOVATIVE PROPERTIES COMPANY, St. Paul, MN (US)

(72) Inventors: Bryan T. Whiting, St. Paul, MN (US); Kevin M. Lewandowski, Inver Grove Heights, MN (US); James A. Bergman, St. Paul, MN (US); Peter M. Simone, Woodbury, MN (US)

(21) Appl. No.: 18/269,727

(22) PCT Filed: Dec. 23, 2021
(86) PCT No.: PCT/IB2021/062266

§ 371 Date: Jun. 26, 2023

Related U.S. Application Data
(60) Provisional application No. 63/132,635, filed on Dec. 31, 2020.

(51) Int. Cl. C09J 11/06; C07C 275/10; C07C 275/14; C08F 220/18; C09J 7/24; C09J 7/25; C09J 7/38; C09J 133/08

(52) U.S. Cl. CPC C09J 11/06; C07C 275/10; C07C 275/14; C08F 220/1808; C09J 7/245; C09J 7/255; C09J 7/385; C09J 133/08; C09J 2301/124; C09J 2301/302; C09J 2301/408; C09J 2301/416; C09J 2427/006; C09J 2433/00; C09J 2467/006

(57) ABSTRACT

Acrylic pressure-sensitive adhesive (“PSA”) compositions crosslinked with a small-molecule crosslinker containing a single, thermally reversible moiety represented by the structure (I) (Formula I)). wherein each R¹ is independently —H or —CH₃, each X is independently C₂-C₆ alkyl, and each R² is independently a C1-C6 alkyl group or an aromatic group. Under high temperature conditions the crosslinks may be broken, thus allowing for good coating and dispensing of the PSA. Upon cooling, the crosslinks reform to create a gelled PSA network without need of additional post-processing curing steps.

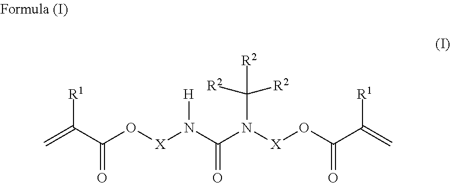

### TECHNICAL FIELD

Crosslinkable compositions, articles containing these compositions, and methods of making the articles are described.

### BACKGROUND

According to the Pressure-Sensitive Tape Council, pressure-sensitive adhesives (“PSAs”) are known to possess properties including the following: (1) aggressive and permanent tack, (2) adherence with no more than finger pressure, (3) sufficient ability to hold onto an adherend, and (4) sufficient cohesive strength to be removed cleanly from the adherend. Materials that have been found to function well as PSAs include polymers designed and formulated to exhibit the requisite viscoelastic properties resulting in a desired balance of tack, peel adhesion, and shear holding power. PSAs are characterized by being normally tacky at room temperature (e.g., 20° C.). Materials that are merely sticky or adhere to a surface do not constitute a PSA; the term PSA encompasses materials with additional viscoelastic properties.

These requirements for pressure-sensitive adhesives are assessed generally by means of tests which are designed to individually measure tack, adhesion (i.e., peel strength), and cohesion (i.e., shear holding power), as noted by A. V. Pocius in Adhesion and Adhesives Technology: An Introduction, 2^{nd} Ed., Hanser Gardner Publication, Cincinnati, Ohio, 2002. These measurements taken together constitute the balance of properties often used to characterize a PSA.

One important class of pressure-sensitive adhesives include those with a (meth)acrylate copolymer as the elastomeric material. The (meth)acrylate copolymers can be used alone or can be combined with tackifiers to provide the desired adhesive properties. Tackifiers can be added, for example, to alter the rheology and compliance of the adhesive composition, to change the surface energy of the adhesive composition, and to alter the melt processing characteristics of the adhesive composition.

### SUMMARY

Disclosed herein are acrylic pressure-sensitive adhesive (“PSA”) compositions crosslinked with a small-molecule crosslinker containing a single, thermally reversible moiety. Under high temperature conditions (e.g., extrusion, hot-melt dispensing) the crosslinks are broken, thus allowing for good coating and dispensing of the PSA. Upon cooling, the crosslinks reform to create a gelled PSA network without need of additional post-processing curing steps.

In one aspect, provided is a small-molecule crosslinker represented by the structure (I)

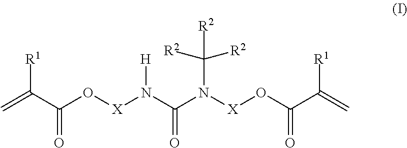

wherein
each R¹ is independently —H or —CH₃,
each X is independently C₂-C₆ alkyl, and
each R₂ is independently a C₁-C₆ alkyl group or an aromatic group.

In another aspect, provided is a pressure-sensitive adhesive including the small-molecule crosslinker represented by the structure (I) and methods of preparing same.

In another aspect, provided are articles including pressure-sensitive adhesives including the small-molecule crosslinker represented by the structure (I).

As used herein, the term “(meth)acrylate” refers to either a methacrylate or acrylate. In many embodiments, the (meth)acrylate is an acrylate. These monomers have a (meth)acryloyl group of formula CH₂═CR—(CO)— where R is hydrogen or methyl.

Features and advantages of the present disclosure will be further understood upon consideration of the detailed description as well as the appended claims.

### DETAILED DESCRIPTION

Acrylic pressure sensitive adhesives (“PSAs”) have advantages over rubber PSAs such as, for example, exhibiting high tack without need of an added tackifier and high chemical stability. One drawback of acrylics, however, is the necessity of covalent crosslinking after coating to achieve necessary cohesive strengths for most applications. Multiblock rubbers on the other hand, can achieve high cohesive strength with no post-cure, streamlining their manufacturing in extruded PSA (e.g., hot-melt) applications. Additionally, because the method of crosslinking does not rely on radiative exposure or radical generation, multiblock rubbers are compatible with a wider range of extrudable additives than acrylics.

Dynamic covalent chemistry leverages fast, reversible reactions to exchange covalent bonds under specific conditions. In the case of materials chemistry, this is often the exchange of covalent crosslinks in a networked material, allowing reworkability, recyclability, or self-healing capabilities that is impossible with traditional crosslinks. The present disclosure employs the use of dynamic covalent bonds in the crosslinking moiety to avoid the need to cure acrylic PSAs. Specifically, with acrylic PSAs, reversible crosslinking would allow a fully crosslinked adhesive to ‘de-crosslink’ at elevated temperatures enabling extrusion and coating to a high-quality film. Once coated and cooled to ambient temperatures, this film will ‘re-crosslink’ without additional curing steps.

Dynamic covalent crosslinking typically may occur by either an associative or dissociative mechanism. In the associative mechanism, the crosslinks (or components of the crosslinks) combine to form a metastable intermediate, which then decomposes to swap crosslinks (Scheme 1). However, because of the low crosslinking level in acrylic pressure-sensitive adhesives, the kinetics of this type of mechanism may be too slow for the extrusion timescale.

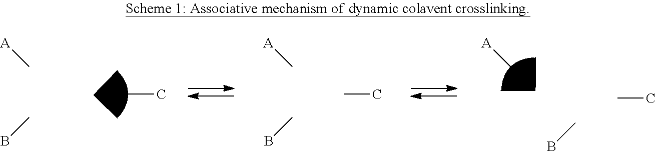

In contrast, a dissociative mechanism works by a simple reversal of the covalent bond to two stable chemical groups (Scheme 2). The dissociative mechanism doesn't rely on overall crosslinking concentration, as each bond can break its covalent linkage without interaction from other crosslinking groups.

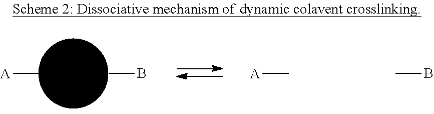

The present disclosure provides a diacrylate crosslinker with a single, bulky asymmetric urea group (Scheme 3) as a crosslinker specifically for acrylic PSAs synthesized in the bulk.

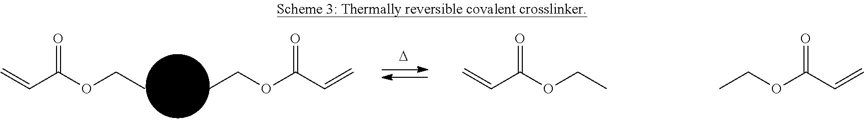

Without the limitations of the two-step solvent polymerization/curing process, this crosslinker can act as a dynamic covalent crosslinker. Furthermore, having a single crosslinking group on the crosslinker molecule may be beneficial from the standpoint of not releasing a small-molecule that could migrate and/or evaporate from the adhesive during high-temperature processing or processing under vacuum, such as during devolatilization to remove residual monomer. Adhesives made using the disclosed crosslinker (Scheme 4) can be extruded into high-quality adhesive films that do not require further crosslinking for clean peel and reasonable shear performance. Both untackified and tackified adhesives are exemplified herein.

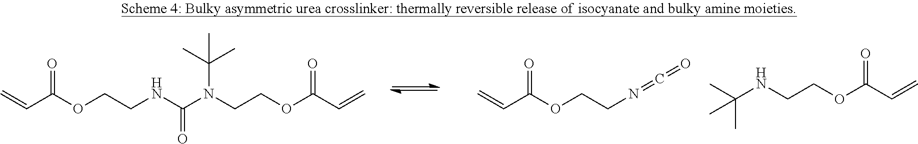

Compared to control adhesives with no crosslinking, adhesives prepared according to the present disclosure may achieve multiple orders of magnitude performance increases in shear, showing the ability of the adhesive to re-form the crosslinks post-extrusion (Scheme 5).

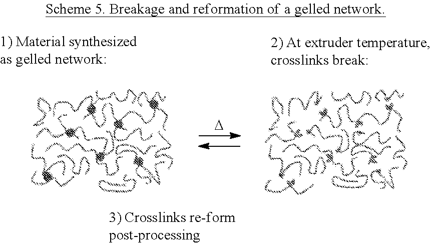

Provided herein is a crosslinker molecule represented by the structure (I)

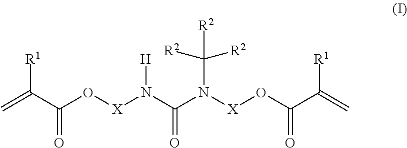

wherein each R¹ is independently —H or —CH₃, each X is independently C₂-C₆ alkyl, and each R² is independently a C₁-C₆ alkyl group or an aromatic group. In some preferred embodiments, R¹ is —H, X is C₂ alkyl, and R² is —CH₃. Such crosslinker molecules may be prepared by methods known to those of ordinary skill in the relevant arts, for example, by reaction of a product formed from the reaction of an isocyanatoalkyl (meth)acrylate (e.g., 2-isocyanatoethyl acrylate) and an alkyl ethanolamine (e.g., N-t-butyl ethanolamine) with a (meth)acryloyl acid chloride (e.g., acryloyl chloride).

Crosslinker molecules represented by the structure (I) can be included in curable compositions that may function as adhesive compositions, such as, for example, pressure-sensitive adhesives (“PSAs”). The PSA may be any type of PSA such as those described in the Handbook of Pressure-Sensitive Adhesives, Ed. D. Satas, 2nd Edition, Von Nostrand Reinhold, New York, 1989 and may be prepared by methods known to those of ordinary skill in the relevant arts. Classes of useful pressure sensitive adhesives include, for example, rubber resin materials such as tackified natural rubbers or those based on synthetic rubbers, styrene block copolymers, polyvinyl ethers, acrylics (including both acrylates and methacrylates), polyurethanes, poly-alpha-olefins, silicone resins, and the like. Combinations of these adhesives can be used.

Pressure sensitive adhesives that may be useful in embodiments of the present disclosure and their preparation are described, for example, in U.S. Pat. No. 4,994,322 (Delgado et al.), U.S. Pat. No. 4,968,562 (Delgado), EP 0 570 515, and EP 0 617 708, U.S. Pat. Nos. 5,296,277 and 5,362,516 (both Wilson et al.), U.S. Pat. No. 5,141,790 (Calhoun et al.), and WO 96/1687 (Keller et al.) Other examples of PSAs are described in U.S. Pat. No. Re 24,906 (Ulrich), U.S. Pat. No. 4,833,179 (Young et al.), U.S. Pat. No. 5,209,971 (Babu et al.), U.S. Pat. No. 2,736,721 (Dester), and U.S. Pat. No. 5,461,134 (Leir et al.). Acrylate-based PSAs include those described in U.S. Pat. No. 4,181,752 (Clemens et al.) and U.S. Pat. No. 4,418,120 (Kealy et al.), WO 95/13331.

In one aspect, provided is a curable composition comprising a first monomer (e.g., a (meth)acrylate monomer), an initiator (e.g., a photoinitiator such as those available under the trade designation OMNIRAD), and a crosslinker represented by the structure (I) wherein, in some preferred embodiments, R¹ is —H, X is C₂ alkyl, and R² is —CH₃. Typically, the curable composition may include 0.05 wt. % to 5 wt. % of the initiator and 0.05 wt. % to 2 wt. % of the crosslinker. In some embodiments, the curable composition may further include a second monomer (e.g., a (meth)acrylate monomer). In preferred embodiments, the composition may be essentially free of protic monomers. As used herein, the term “protic monomer” refers to a monomer having a functional group that includes a labile H⁺, such as, for example, an alcohol, a carboxylic acid, or an amine.

In some embodiments, the curable composition may further include components such as, for example, a chain transfer reagent (e.g., isooctyl thioglycolate), a tackifier (e.g., tackifying resins available commercially under the trade designations FORAL, STAYBELITE, and WINGTACK), and combinations thereof. In some embodiments, the curable composition may include 0.01 wt. % to 5 wt. % of the chain transfer reagent. In some embodiments, the curable composition may include up to 60 wt. %, optionally 5 wt. % to 50 wt. % of the tackifier. Other additives that can be included in embodiments of the present disclosure may be selected from the group consisting of a pigment, a filler, a plasticizer, and combinations thereof. 17.

Pressure-sensitive adhesives prepared according to the present disclosure can be used in a variety of traditional pressure-sensitive adhesive articles, such as, for example, tapes (e.g., single-sided tapes, double-sided tapes), labels, decals, transfer tapes, and other articles. Such articles may be prepared according to techniques known to those of ordinary skill in the relevant arts, for example, by providing a substrate (e.g., a poly(ethylene terephthalate) film) and positioning a curable composition described above adjacent to the substrate.

Objects and advantages of this disclosure are further illustrated by the following non-limiting examples, but the particular materials and amounts thereof recited in these examples, as well as other conditions and details, should not be construed to unduly limit this disclosure.

### EXAMPLES

Unless otherwise noted or readily apparent from the context, all parts, percentages, ratios, etc. in the Examples and the rest of the specification are by weight.

Materials Used in the Examples

| Abbreviation | Description | Source |
|---|---|---|
| C8 Acrylate | Octyl acrylate isomer blend prepared as | 3M Company, St. Paul, MN |
| | described in U.S. Pat. No. 9,102,774 (Clapper | |
| | et al.) | |
| IBOA | Isobornyl acrylate | Sigma-Aldrich (St. Louis, |
| | | MO, USA) |
| DMAA | Dimethyl acrylamide | Tokyo Chemical Industry |
| | | Co., LTD (TCI) (Tokyo, |
| | | Japan) |
| NVP | N-Vinyl pyrrolidone | Tokyo Chemical Industry |
| | | Co., LTD (TCI) (Tokyo, |
| | | Japan) |
| BDK | 2,2-dimethoxy-2-phenylacetophenone, obtained | IGM Resins USA Inc., |
| | under the trade designation “OMNIRAD BDK” | Charlotte, NC |
| IOTG | Isooctyl thioglycolate | Tokyo Chemical Industry |
| | | Co., LTD (TCI) (Tokyo, |
| | | Japan) |
| IOA | Isooctyl acrylate | 3M Company, St. Paul, MN |
| EVA | A clear polyethylene vinyl acetate film, 0.065 | Flint Hills Resources, |
| | millimeters thick, obtained under the trade | Wichita, KS |
| | designation “VA-24” | |
| BA | Butyl acrylate | Sigma-Aldrich (St. Louis, |
| | | MO, USA) |
| MA | Methyl acrylate | Sigma-Aldrich (St. Louis, |
| | | MO, USA) |
| ABP | 4-Acryloyl benzophenone | 3M Company, St. Paul, MN |
| EHA | 2-Ethylhexyl acrylate | BASF Corporation, Houston, |
| | | TX |
| HDDA | Hexanediol diacrylate | Tokyo Chemical Industry |
| | | Co., LTD (TCI) (Tokyo, |
| | | Japan) |
| D1119 | A linear triblock styrene-isoprene-styrene | Kraton Corporation, Houston, |
| | triblock copolymer obtained under the trade | TX |
| | designation “D1119 P” | |
| F85 | Glycerol ester of hydrogenated wood rosin, a | Pinova, Inc. Brunswick, GA |
| | tackifying resin obtained under the trade | |
| | designation “FORAL 85” | |
| CB | Carbon black, a pigment obtained under the | Cabot Corporation, Billerica, |
| | trade designation “EMPEROR 1200” | MA |
| S3E | Ester of hydrogenated rosin, a tackifying resin | Eastman Chemical Company, |
| | obtained under the trade designation | Kingsport, TN |
| | “STAYBELITE ESTER 3E” | |
| W10 | A liquid aliphatic C-5 hydrocarbon, a tackifying | Cray Valley USA, LLC, |
| | resin obtained under the trade designation | Exton, PA |
| | “WINGTACK 10” | |
| RF02N | A 75 micron thick silicone-coated poly(ethylene | SKC, Seoul, South Korea |
| | terephthalate) film, a release liner obtained | |
| | under the trade designation RF02N | |
| RF12N | A 75 micron thick silicone-coated poly(ethylene | SKC, Seoul, South Korea |
| | terephthalate) film, a release liner obtained | |
| | under the trade designation RF12N | |
| 3SAB | A 0.002 inch (51 micrometer) thick primed | Mitsubishi Polyester Film, |
| | poly(ethylene terephthalate) film obtained under | Greer, SC |
| | the trade designation HOSTOPHAN 3SAB | |

### Test Methods

### Test Method 1: 180° Peel Adhesion Test (“Peel”)

Peel adhesion was the force required to remove an adhesive-coated test specimen from a test panel measured at a specific angle and rate of removal. In the Examples, this force is expressed in ounces per inch width of coated sheet. The following procedure was used:

Peel adhesion strength was measured at a 180° peel angle using an IMASS SP-200 slip/peel tester (available from IMASS, Inc., Accord MA) at a peel rate of 305 mm/minute (12 inches/minute). Stainless steel (SS) test panels were cleaned with methyl ethyl ketone and a clean KIMWIPE tissue (Kimberly-Clark) three times. The cleaned panel was dried at room temperature. Polypropylene (PP) test panels were wiped with a dry KIMWIPE tissue to remove dust and then used directly. The adhesive was laminated to 3SAB and allowed to dwell for 24 hours. The adhesive coated film was cut into tapes measuring 1.27 cm×20 cm (½ in.×8 in.). A test sample was prepared by rolling the tape down onto a cleaned panel with 3 passes of a 2.0 kg (4.5 lb.) rubber roller. The prepared samples were dwelled at 23° C./50% RH for 0 to 15 minutes before testing. One-two samples were tested for each example. The resulting peel adhesion was converted from ounces/0.5 inch to ounces/inch. The failure mode was also recorded for each peel sample.

### Test Method 2: Shear Test Method

Stainless steel (SS) plates were prepared for testing by cleaning with methyl ethyl ketone and a clean KIMWIPE tissue three times. The adhesive was laminated to 3SAB and allowed to dwell for 24 hours. The adhesive films described were cut into strips (1.27 cm in width) and adhered by their adhesive to flat, rigid stainless-steel plates with exactly 2.54 cm length of each adhesive film strip in contact with the plate to which it was adhered. A weight of 2 kilograms (4.5 pounds) was rolled over the adhered portion with three passes. Each of the resulting plates with the adhered film strip was equilibrated at room temperature for 60 minutes. Afterwards, the samples were transferred to a room with 23° C./50% relative humidity, in which a 500 g weight was hung from the free end of the adhered film strip with the panel tilted 2° from the vertical to insure against any peeling forces. The time (in minutes) at which the weight fell, as a result of the adhesive film strip releasing from the plate, was recorded. The test was discontinued at 10,000 minutes if there was no failure. In the Tables, this is designated as 10,000+ minutes. Two specimens of each tape (adhesive film strip) were tested and the shear strength tests were averaged to obtain the reported shear values. Additionally, for samples that did not hang for 10,000+ minutes, the failure modes of the samples were recorded.

### Test Method 3: Frequency Sweep Dynamic Mechanical Analysis Test Method and Crosslinking Factor/Crossover Frequency

Version A: 0.04 rad/s to 400 rads at 70° C.

Dynamic Mechanical Analysis (DMA) was completed using an AR2000ex parallel plate rheometer (TA Instruments, New Castle, DE) equipped with an 8 millimeter diameter stainless steel parallel plate fixture and an environmental test chamber A section of adhesive about 2 inches (5.08 centimeter) by 4 inches (10.16 centimeter) was cut from each sample that was coated between release liners. The samples were then folded and laminated on release liner using a silicone roller until an adhesive film about 1 millimeter thick was achieved. After lamination, an 8 millimeter diameter die was used to obtain the test specimen. The test specimen was then centered on the bottom plate in the rheometer chamber and the instrument controls were used to lower the top plate onto the specimen so that the edges of the sample were uniform with the edges of the top and bottom plates. The environmental test chamber of the instrument was then closed, and the temperature was equilibrated to 70° C. for a period of one minute. The temperature was maintained at 70° C. for the duration of the test. The specimen was oscillated in a logarithmic sweep over the angular frequency range of 0.04 radians/second to 400 radians/second at a constant strain of 3%. Data was collected at the rate of 5 data points per angular frequency decade. Each angular frequency was allowed 3 seconds of equilibration and 3 seconds of sampling.

To quantify each sample's crosslinking, the crosslinking factor was defined as the ratio of tan(delta) at 0.25 radians/second to tan(delta) at 63.4 radians/second. Additionally, each sample was analyzed to determine if a crossover frequency was present, where below a critical frequency the loss modulus (G″) exceeded the storage modulus (G′) indicating a system with greater viscous character than elastic character.

### Test Method 4: Molecular Weight Analysis Using GPC

The molecular weight distribution of the copolymers was characterized using conventional gel permeation chromatography (GPC). The GPC instrumentation, which was obtained from Waters Corporation (Milford, MA), included a high pressure liquid chromatography pump (Model 1515HPLC), an auto-sampler (Model 717), a UV detector (Model 2487), and a refractive index detector (Model 2410). The chromatograph was equipped with two 5 micron PLgel MIXED-D columns, available from Varian Inc. (Palo Alto, CA).

Samples of polymeric solutions were prepared by dissolving polymer or dried polymer samples in tetrahydrofuran at a conclentration of 0.5 percent (weight/volume) and filtering through a 0.2 micron polytetrafluoroethylene filter that is available from VWR International (West Chester, PA). The resulting samples were injected into the GPC and eluted at a rate of 1 milliliter per minute through the columns maintained at 35° C. The system was calibrated with polystyrene standards using a linear least squares fit analysis to establish a calibration curve. The weight average molecular weight (Mw) was calculated for each sample against this standard calibration curve.

### Test Method 5: Melt Viscosity Analysis

The melt viscosity of the uncured polymers was taken as the complex viscosity measured via oscillatory shear. To prepare samples for testing 5-10 g of polymer were pressed between release liners in a heated press at 150° C. for 5 min. To control the film thickness, 250 μm (10 mil) shims were used in the press. The pressed polymer samples were layered (removing liners between layers) to a thickness of 1 mm, and test samples were punched from these laminated stacks. A circular sample with a diameter of 20 mm was punched from the uncured layered coatings and analyzed on a DHR-3 rheometer equipped with a flat 20 mm spindle and a Peltier plate for temp control. The viscosity was measured at a frequency of 1.0 rad/s with a strain of 10% as the temperature stepped from 90° C. to 190° C. in 20° C. increments.

### Test Method 6: Creep Strain Rate Analysis

Polymer film samples for testing were prepared in an identical manner as described in the melt viscosity analysis method. For creep strain rate (CSR) testing a circular sample with a diameter of 8 mm was punched from the hot-pressed films which had been layered to a final thickness of 1 mm. The samples were analyzed on a TA Instruments DHR-3 rheometer equipped with a flat 8 mm spindle and a Peltier plate for temp control. The sample was subject to an 8 kPa shear stress and the strain percentage is measured over time at 25° C. The creep strain rate is taken as the change in strain percent between 20 and 30 minutes during the creep test.

### Test Method 7: Dynamic Mechanical Analysis

Polymer film samples for testing were prepared in an identical manner as described in the melt viscosity analysis method. For dynamic mechanical analysis (DMA) testing 250 μm (10 mil) thick hot-pressed films were crosslinked by irradiating each side of the sample through the release liner in a Fusion UV processor equipped with a D-bulb. The system settings were selected to provide a total UVA energy dosage of 1.5 J/cm2, as calibrated using an EIT Power Puck II UV radiometer with a piece of the release liner over the sensor. The crosslinked polymer samples were layered (removing liners between layers) to a thickness of 1 mm, and DMA samples were punched from these laminated stacks. The samples were analyzed in shear mode on a DHR-3 rheometer from TA Instruments (New Castle, DE) equipped with 8 mm parallel plates. The DMA samples were analyzed via oscillatory shear while ramping temperature between −30° C. and 180° C. at 3° C./min, with a frequency of 1 Hz. The strain was 2% for temperatures below 35° C., and 5% for temperatures above 35° C. The storage modulus (G′) at 25° C. and 85° C., and tan δ (G″/G′) at 85° C. were taken from the temperature ramp data.

### EXAMPLE PREPARATIONS

### Preparatory Example 1: 2-[[tert-butyl(2-prop-2-enoyloxyethyl)carbamoyl]amino]ethyl prop-2-enoate (“M1”)

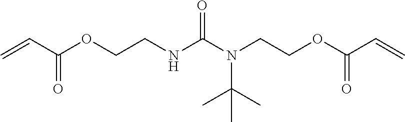 [SMILES: C=CC(=O)OCCNC(=O)N(CCOC(=O)C=C)C(C)(C)C]

2-Isocyanatoethyl acrylate (108.38 g, 768 mmol, Karenz AOI, Showa Denko, JP) was added dropwise to a solution of 90.00 g (768 mmol) of N-t-butyl ethanolamine (90.00 g, 768 mmol, Oakwood Chemical, Estill, SC) and ethyl acetate (300 mL) at room temperature. After 1 hour of stirring at room temperature, the solvent was removed under vacuum to give 2-[[t-butyl(2-hydroxyethyl)carbamoyl]amino]ethyl prop-2-enoate as a thick, slightly orange oil (197.55 g).

A mixture of 2-[[t-butyl(2-hydroxyethyl)carbamoyl]amino]ethyl prop-2-enoate (100 g, 0.39 mol), triethylamine (39.17 g, 0.39 mol, Alfa Aesar, Ward Hill, MA), and dichloromethane (1 L) was cooled in an ice bath. Acryloyl chloride (35.03 g, 39 mol, Alfa Aesar) was added dropwise over 30 minutes. After stirring for two hours, the solvent was removed under vacuum. Ethyl acetate (500 mL) was added, and the mixture was washed with 1.0 M HCl followed by saturated sodium bicarbonate. The organic layer was dried over anhydrous magnesium sulfate and filtered. The solvent was removed under vacuum to give M1 as a yellow oil. (78.89 g).

### Preparatory Example 2: Comparative Diurea-Diacrylate Crosslinker CM-1

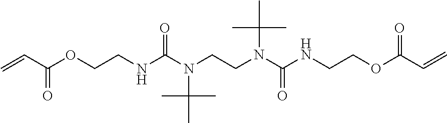 [SMILES: C=CC(=O)OCCNC(=O)N(CCN(C(=O)NCCOC(=O)C=C)C(C)(C)C)C(C)(C)C]

A solution of 2-isocyanatoethyl acrylate (8.19 g, 58 mmol, Karenz AOI) and ethyl acetate (10 mL) was added dropwise to a solution of N,N′-di-tertbutyl ethylene diamine (5.00 g, 29 mmol) and ethyl acetate (30 mL) at room temperature. After 30 minutes, the solvent was removed under vacuum to give CM-1 as a white solid (13.19 g).

### Preparatory Example 3: Comparative Diurea-Dimethacrylate Crosslinker CM-2

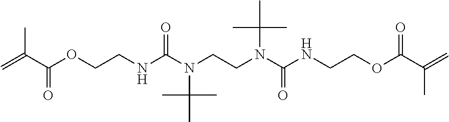 [SMILES: C=C(C)C(=O)OCCNC(=O)N(CCN(C(=O)NCCOC(=O)C(=C)C)C(C)(C)C)C(C)(C)C]

A solution of 2-isocyanatoethyl methacrylate (9.00 g, 58 mmol, Alfa Aesar) and ethyl acetate (10 mL) was added dropwise to a solution of N,N′-di-tertbutyl ethylene diamine (5.00 g, 29 mmol) and ethyl acetate (30 mL) at room temperature. After 30 minutes, the solvent was removed under vacuum to give CM-2 as a white solid (13.90 g).

### Preparatory Example 4: Comparative Diurea-Dimethacrylate Crosslinker CM-3

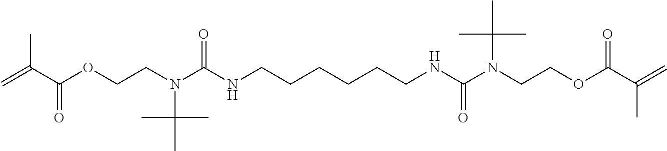 [SMILES: C=C(C)C(=O)OCCN(C(=O)NCCCCCCNC(=O)N(CCOC(=O)C(=C)C)C(C)(C)C)C(C)(C)C]

t-Butyl aminoethyl methacrylate (6.61 g, 36 mmol, Sigma-Aldrich Corp, St. Louis, MO) was added dropwise to a solution of 1,6-hexanediol diisocyanate (3.00 g, 18 mmol, Alfa Aesar) and ethyl acetate (30 mL) while the solution was cooled in an ice bath. After full addition, the solvent was removed under vacuum to give CM-3 as a thick yellow oil (9.60 g).

### Comparative Example 1 (CE1) and Examples 1-3 (EX1-EX3): Preparation of Adhesive Films and Testing Results

To prepare the polymers: monomers, crosslinker, chain-transfer reagent, and photoinitiator shown in Table 1 were mixed until homogeneous. 16 g of this formulation was added to EVA film heat sealed on three sides. Entrained air was displaced, and the fourth side was sealed. This sealed mixture was submerged in a 22° C. water bath and exposed to UV irradiance with a peak emission of 350 nm for 8 minutes, at which point the samples were flipped and further irradiated for 8 minutes.

To form films: polymerized samples were added to an MC-5 Twin-Screw Microcompounder (Xplore Instruments) at a temperature between 150-160° C. at a screw speed of 60 RPM. The samples were compounded for 3 or 5 minutes then dispensed onto release liner. To form the extruded materials into films, the sample was pressed using a heated hydraulic press at 10,000 PSI for 3 minutes at 160° C. between release liners to approximately 3 mil thickness. The films were rested at room temperature for at least 24 hours before testing. The shear performance of the samples was measured, and results are shown in Table 2.

TABLE 1
Formulations of Microcompounder-Formed Films

| | C8 | | | | | |
|---|---|---|---|---|---|---|
| | Acrylate | DMAA | IBOA | M1 | IOTG | BDK |
| | (wt. %) | (wt. %) | (wt. %) | (wt. %) | (wt. %) | (wt. %) |
| CE-1 | 84.85 | 4.99 | 9.98 | 0.00 | 0.03 | 0.15 |
| EX-1 | 84.72 | 4.98 | 9.97 | 0.15 | 0.03 | 0.15 |
| EX-2 | 84.54 | 4.97 | 9.95 | 0.30 | 0.10 | 0.15 |
| EX-3 | 84.59 | 4.98 | 9.95 | 0.30 | 0.03 | 0.15 |

TABLE 2
Shear Results of Microcompounder-Formed Films

| | Shear (Minutes) | Failure Mode |
|---|---|---|
| CE-1 | 6 | Cohesive |
| EX-1 | 86.5 | Cohesive |
| EX-2 | 876.5 | Cohesive |
| EX-3 | 9126 | Pop-off |

The thermally reversible crosslinkers greatly improve the shear performance of the adhesive films.

### Examples 4-6 (EX4-EX6): Preparation of Adhesive Films and Testing Results

To prepare the polymers: monomers, crosslinker, chain-transfer reagent, and photoinitiator shown in Table 3 were mixed until homogeneous. 16 g of this formulation was added to EVA film heat sealed on three sides. Entrained air was displaced, and the fourth side was sealed. This sealed mixture was submerged in a 22° C. water bath and exposed to UV irradiance with a peak emission of 350 nm for 8 minutes, at which point the samples were flipped and further irradiated for 8 minutes.

To form films: polymerized samples and additives were added to an MC-5 Twin-Screw Microcompounder (Xplore Instruments) at a temperature between 150-160° C. at a screw speed of 60 RPM. The samples were compounded for 3 or 5 minutes then dispensed onto release liner. To form the extruded materials into films, the sample was pressed using a heated hydraulic press at 10,000 PSI for 3 minutes at 160° C. between release liners to approximately 3 mil thickness. The films were rested at RT for at least 24 hours before testing.

The peel performance from stainless steel and polypropylene substrates and the shear performance of the samples was measured, and results are shown in Table 4.

TABLE 3
Formulation of Microcompounder-Formed Films with Additives

| | IOA | NVP | M1 | IOTG | BDK | F85 | D1119 |
|---|---|---|---|---|---|---|---|
| | (wt. %) | (wt. %) | (wt. %) | (wt. %) | (wt. %) | (wt. %) | (wt. %) |
| EX-4 | 86.11 | 4.49 | 0.27 | 0.03 | 0.13 | 8.97 | — |
| EX-5 | 79.03 | 4.12 | 0.25 | 0.02 | 0.12 | 16.46 | — |
| EX-6 | 71.31 | 3.71 | 0.33 | 0.02 | 0.11 | 24.51 | — |
| EX-7 | 79.03 | 4.12 | 0.25 | 0.02 | 0.12 | — | 16.46 |

TABLE 4
Peel and Shear Results of Microcompounder-
Formed Films with Additives

| | SS Peel | Failure | PP Peel | Failure | Shear | Failure |
|---|---|---|---|---|---|---|
| | (oz/in) | Mode | (oz/in) | Mode | (minutes) | Mode |
| EX-4 | 30.4 | Clean peel | 18.5 | Clean | 1319 | Pop-off |
| EX-5 | 74.3 | Mixed | 45.1 | Residue | 87.5 | Cohesive |
| EX-6 | 63.0 | Clean peel | 33.0 | Clean | 213 | Mixed |
| EX-7 | 35.1 | Clean peel | 25.8 | Clean | 10,000+ | — |

The thermally reversible crosslinkers drastically improves the shear performance and cohesion (as seen in the transition from cohesive failure to clean peel) of the resulting adhesive compositions, even in the presence of additives that can prevent cure in standard UV- or e-beam-cured compositions.

### Comparative Example 2 (CE2) and Examples 8-9 (EX8-EX9)

To prepare the polymers: monomers, crosslinker, chain-transfer reagent, and photoinitiator shown in Table 5 were mixed until homogeneous. 25 g of this formulation was added to EVA film heat sealed on three sides. Entrained air was displaced, and the fourth side was sealed. This sealed mixture was submerged in a 22° C. water bath and exposed to UV irradiance with a peak emission of 350 nm for 8 minutes, at which point the samples were flipped and further irradiated for 8 minutes.

To form films: the polymerized materials were added to a batch-mode, twin-screw extruder and compounded at a temperature between 150-160° C. at a screw speed of 300 RPM for 3 minutes. The samples were coated using a screw speed of 100 RPM and 150° C. using a contact die onto release liner at approximately 3 mil thickness. The films were rested at 22° C. for at least 24 hours before testing.

The peel performance from stainless steel and polypropylene substrates and the shear performance of the samples was measured, and results are shown in Table 6.

TABLE 5
Formulation of Extruded Films

| | C8 | | | | | |
|---|---|---|---|---|---|---|
| | Acrylate | DMAA | IBOA | M1 | IOTG | BDK |
| | (wt. %) | (wt. %) | (wt. %) | (wt. %) | (wt. %) | (wt. %) |
| CE-2 | 84.85 | 4.99 | 9.98 | 0.00 | 0.03 | 0.15 |
| EX-8 | 84.59 | 4.98 | 9.95 | 0.30 | 0.03 | 0.15 |
| EX-9 | 84.47 | 4.97 | 9.94 | 0.45 | 0.03 | 0.15 |

TABLE 6
Peel and Shear Results of Extruded Films

| | SS Peel | Failure | PP Peel | Failure | Shear | Failure |
|---|---|---|---|---|---|---|
| | (oz/in) | Mode | (oz/in) | Mode | (minutes) | Mode |
| CE-2 | 129.3 | Mixed/2- | 23.2 | Residue | 5 | Cohesive |
| | | bond | | | | |
| EX-8 | 32.4 | Clean peel | 7.8 | Clean | 1552 | Pop-off |
| EX-9 | 17.4 | Clean peel | 4.7 | Clean | 5298 | Pop-off |

The thermally reversible crosslinkers drastically improves the shear performance of the resulting adhesive compositions after extrusion

### Comparative Example 3 (CE3) and Examples 10-13 (EX10-EX13)

To prepare the polymers: monomers, crosslinker, chain-transfer reagent, and photoinitiator shown in Table 7 were mixed until homogeneous. 25 g of this formulation was added to EVA film heat sealed on three sides. Entrained air was displaced, and the fourth side was sealed. This sealed mixture was submerged in a 22° C. water bath and exposed to UV irradiance with a peak emission of 350 nm for 8 minutes, at which point the samples were flipped and further irradiated for 8 minutes.

To form films: the polymerized materials and additives were added to a batch-mode, twin-screw extruder and compounded at a temperature of 150° C. at a screw speed of 300 RPM for 3 minutes. The samples were coated using a screw speed of 100 RPM and 150° C. using a contact die onto release liner at approximately 4 mil thickness. The films were rested at 22° C. for at least 24 hours before testing.

The peel performance from stainless steel and the shear performance of the samples was measured, and results are shown in Table 8.

TABLE 7
Formulation of Extruded Films with Additives

| | IOA | NVP | IBOA | M1 | IOTG | BDK | S3E | W10 | CB |
|---|---|---|---|---|---|---|---|---|---|
| | (wt. %) | (wt. %) | (wt. %) | (wt. %) | (wt. %) | (wt. %) | (wt %) | (wt %) | (wt %) |
| CE-3 | 84.85 | 4.99 | 9.98 | 0.00 | 0.03 | 0.15 | 0.00 | 0.00 | 0.00 |
| EX-10 | 84.59 | 4.98 | 9.95 | 0.30 | 0.03 | 0.15 | 0.00 | 0.00 | 0.00 |
| EX-11 | 80.54 | 4.74 | 9.48 | 0.28 | 0.03 | 0.14 | 4.74 | 0.00 | 0.00 |
| EX-12 | 80.54 | 4.74 | 9.48 | 0.28 | 0.03 | 0.14 | 0.00 | 4.74 | 0.00 |
| EX-13 | 80.54 | 4.74 | 9.48 | 0.28 | 0.03 | 0.14 | 0.00 | 0.00 | 4.74 |

TABLE 8
Peel and Shear Results of Extruded Films with Additives

| | SS Peel | | Shear | |
|---|---|---|---|---|
| | (oz/in) | Failure Mode | (minutes) | Failure Mode |
| CE-3 | 74.6 | Cohesive | 2 | Cohesive |
| EX-10 | 30.5 | Clean peel | 559 | Pop-off with residue |
| EX-11 | 17.0 | Clean peel | 405 | Ghost residue |
| EX-12 | 38.2 | Clean peel | 1308 | Ghost residue |
| EX-13 | 50.7 | Clean peel | 386 | Mixed Cohesive/Adhesive |

The thermally reversible crosslinkers drastically improves the shear performance and cohesion (as seen in the transition from cohesive failure to clean peel) of the resulting adhesive compositions, even in the presence of additives that can prevent cure in standard UV- or e-beam-cured compositions.

### Comparative Examples 4-6 (CE4-CE6) and Example 14 (EX14)

95 g EHA and 5 g DMAA were combined with 0.03 g IOTG and 0.10 g BDK and placed in a glass vessel. The mixtures were purged with nitrogen for five minutes then exposed to a UVP XX-15L 15 Watt blacklight with peak emission at 365 nm (Analytik Jena US, Upland, CA) at a distance of 10 centimeters from the lamp with mixing until a polymeric syrup having a Brookfield viscosity of 100 to 5000 centiPoise was formed, at which point the samples were purged with air to quench further polymerization. 10 g of the coatable composition was added to a new vessel and crosslinker and additional photoinitiator were added to the vessel to reach the proportions shown in Table 9 and mixed for at least one hour. These compositions were then knife coated between RF02N and RF12N release liners with a gap of 0.002 inches (51 microns). The coated compositions were irradiated for five minutes using UVA lamps (OSRAM SYLVANIA F40/350BL BLACKLIGHT, peak wavelength of 352 nanometers, 40 Watts) to provide total UVA energy of 1500 milliJoules/square centimeter.

Cured films of the crosslinked adhesive on release liner were secured with tape to an aluminum plate. The plate was loaded into a Precision Scientific vacuum oven (GCA Corporation) equipped with an RV-5 vacuum pump (Edwards Vacuum) set at 160° C. Vacuum was applied to the oven for 30 minutes. The samples were then removed from the oven and allowed to rest at 22° C. for at least 24 hours before testing. Films were tested by frequency sweep DMA for crosslinking factor and crossover frequency before and after devolatilization as shown in Table 10.

TABLE 9
Formulation of Films in Devolatilization Study (Polymerization Method 3)

| | EHA | DMAA | BDK | CM-1 | CM-2 | CM-3 | M1 |
|---|---|---|---|---|---|---|---|
| | (wt %) | (wt %) | (wt %) | (wt %) | (wt %) | (wt %) | (wt %) |
| CE-4 | 94.52 | 4.97 | 0.20 | 0.30 | 0.00 | 0.00 | 0.00 |
| CE-5 | 94.50 | 4.97 | 0.20 | 0.00 | 0.32 | 0.00 | 0.00 |
| CE-6 | 94.47 | 4.97 | 0.20 | 0.00 | 0.00 | 0.36 | 0.00 |
| EX-14 | 94.61 | 4.98 | 0.20 | 0.00 | 0.00 | 0.00 | 0.21 |

TABLE 10
Crosslinking Factors of Films in Devolatilization Study

| | Before Devolatilization | | After Devolatilization | | |
|---|---|---|---|---|---|
| | Cross- | Crossover | Cross- | | Crossover |
| | linking | Frequency | linking | Percent | Frequency |
| | Factor | Observed | Factor | Change | Observed |
| CE-4 | 0.79 | No | 0.31 | −61.4% | Yes |
| CE-5 | 0.77 | No | 0.36 | −53.4% | Yes |
| CE-6 | 0.63 | No | 0.36 | −42.0% | Yes |
| EX-14 | 0.60 | No | 0.63 | 3.8% | No |

During the processing of adhesive films, it can be advantageous to expose the materials to a devolatilization process involving heat and/or vacuum and/or a purging gas. This devolatilization decreases the volatile organic components remaining from the production process that otherwise lead to odor, environmental release, and safety concerns. However, because of the thermally reversible nature of bulky urea bonds, under these conditions it is possible for the crosslinks to reverse. As shown in Table 10, di-functional urea crosslinkers (CE-4-CE-6) of the type described in US20170327627 lose their efficiency under these conditions, as the central crosslinking moiety can be lost along with the other volatile organic, as evidenced by the decrease is Crosslinking Factor and observation of a crossover frequency in these samples. The monofunctional crosslinker M1, however, does not show any loss of Crosslinking Factor or have an observed crossover frequency after devolatilization This is likely because all components of the crosslinker remain covalently bound to polymer chains even during thermal reversion.

### COMPARATIVE EXAMPLES 7-9 (CE7-CE9) AND EXAMPLES 15-17 (EX15-EX17)

The monomers, along with photoinitiator, crosslinker, and chain transfer agent shown in Table 11 were combined and then added to a pouch of EVA film sealed on 3 sides. Air was displaced from the sample and the open end was heat-sealed. The samples were placed in a rack that held them at a thickness of approximately 0.3 inches. The polymerizations were initiated by placing the rack containing the pouches under 405 nm LED lights. The rack and pouches were submerged in a circulating cooling water bath set at 16° C. during the polymerization to control the temperature. The light exposure conditions for the polymerization were as follows. The polymerizations were started with a LED light power setting of 30% for the first 9 minutes. The light power was increased to 40% for an additional 10 minutes. Finally, the rack holding the samples was flipped 180° so the bottom side of the samples was irradiated at the 40% power setting for an additional 6 minutes. The average UVV irradiation power at different locations under the LED lights were calibrated with an EIT power puck radiometer.

These materials were characterized by Test Methods 4-7 and the results are shown in Tables 11 and 12.

TABLE 11
Formulations of Flowable Adhesives

| | BA | MA | ABP | M1 | IOTG | Mw |
|---|---|---|---|---|---|---|
| | (wt %) | (wt %) | (pph) | (pph) | (pph) | (kDa) |
| CE-7 | 60 | 40 | 0.2 | 0.0 | 0.06 | 510 |
| CE-8 | 60 | 40 | 0.2 | 0.0 | 0.1 | 335 |
| CE-9 | 60 | 40 | 0.2 | 0.0 | 0.15 | 285 |
| EX-15 | 60 | 40 | 0.2 | 0.3 | 0.2 | N/A |
| EX-16 | 60 | 40 | 0.3 | 0.3 | 0.25 | N/A |
| EX-17 | 60 | 40 | 0.3 | 0.3 | 0.3 | N/A |

TABLE 12
Material Testing Properties of Flowable Adhesives

| | 170° C. Melt | | | | |
|---|---|---|---|---|---|
| | Viscosity | CSR | G′ at 25° C. | G′ at 85° C. | Tan(delta) |
| | (Pa · s) | (%/s) | (kPa) | (kPa) | at 85° C. |
| CE-7 | 4580 | 0.041 | 156 | 70 | 0.32 |
| CE-8 | 1990 | 0.066 | 148 | 55 | 0.38 |
| CE-9 | 750 | 0.247 | 133 | 32 | 0.59 |
| EX-15 | 940 | 0.014 | 127 | 42 | 0.32 |
| EX-16 | 400 | 0.028 | 130 | 39 | 0.34 |
| EX-17 | 170 | 0.095 | 122 | 28 | 0.44 |

It has been observed that the urea diacrylate crosslinker can be used to reduce polymer melt viscosity without compromising polymer physical properties. Examples EX-15 through E-17 incorporate the urea diacrylate crosslinker, M1 and have lower melt viscosity than comparative examples CE-7 through CE-9. Despite lower melt viscosities, EX-15 through E-17 still maintain desirably low CSR and tan(delta) at 85° C. In order to achieve similar values for CSR and tan(delta) at 85° C., polymers not incorporating the urea diacrylate crosslinker must have higher molecular weight (CE-7 and CE-8). However, higher polymer molecular weight results in high melt viscosity, which is challenging for hot melt extrusion processing of thin PSA films. These characteristics of low melt viscosity and low CSR are particularly advantageous for melt extrusion coated, optically clear PSAs utilized in electronic display lamination For these applications the PSA is typically supplied in the die-cut shape of the display screen, so low CSR is needed to maintain dimensional stability (resist flow or sagging) at the edges of the die-cut. Additionally, lower melt viscosity facilitates achieving high clarity, defect free PSA films via melt extrusion coating.

### Comparative Example 10 (CE-10) and Example 18 (EX-18)

95 g EHA and 5 g NVP were combined with 0.04 g BDK and placed in a glass vessel. The mixtures were purged with nitrogen for 5 minutes then exposed to an UVP XX-15L 15 Watt blacklight with peak emission at 365 nm (Analytik Jena US, Upland, CA) at a distance of 10 centimeters from the lamp with mixing until a polymeric syrup having a Brookfield viscosity of 100 to 5000 centiPoise was formed, at which point the samples were purged with air to quench further polymerization. 10 g of the coatable composition was added to a new vessel and crosslinker and additional photoinitiator were added to the vessel to reach the proportions shown in Table 13 and mixed for at least one hour. These compositions were then knife coated between 3SAB film and RF12N release liner with a gap of 0.002 inch (51 microns). The coated compositions were irradiated for five minutes using UVA lamps (OSRAM SYLVANIA F40/350BL BLACKLIGHT, peak wavelength of 352 nanometers, 40 Watts) to provide total UVA energy of 1500 milliJoules/square centimeter.

The cured adhesive films were allowed to dwell for 24 hours. The adhesive coated film was cut into tapes measuring 1.27 cm×20 cm (½ in.×8 in.). Samples were attached to substrates by heat-lamination The samples were laminated onto a substrate at 140° C. at a speed of 0.3 meters per minute using a LINEA DH-360 heated roll-laminator (Vivid Laminating Technologies, LLC, Ashby-de-la-Zouch, United Kingdom). Substrates were laminated to polycarbonate (PC) test panels wiped with a KIMWIPE and used directly on SUPER POLX 1200 knitted polyester sheets (POLY) (Berkshire Corp., Great Barrington, MA). The polyester sheets were then attached to stainless steel peel testing plates by double-sided tape. The heat-laminated samples were dwelled at 22° C. for 24 hours before testing. Peel adhesion strength was measured at a 180° peel angle using an IMASS SP-200 slip/peel tester (available from IMASS, Inc., Accord MA) at a peel rate of 305 mm/minute (12 inches/minute). Two samples were tested for each example. The resulting peel adhesion was converted from ounces/0.5 inch to ounces/inch. The results of the peel testing are shown in Table 14.

TABLE 13
Formulations of Heat-laminated Adhesives

| | EHA | NVP | BDK | HDDA | Ml |
|---|---|---|---|---|---|
| | (wt %) | (wt %) | (wt %) | (wt %) | (wt %) |
| CE-10 | 94.67 | 4.98 | 0.15 | 0.20 | 0.00 |
| EX-18 | 94.59 | 4.98 | 0.15 | 0.00 | 0.28 |

TABLE 14
Peel adhesion of heat-laminated adhesives

| | PC Peel | | POLY Peel | |
|---|---|---|---|---|
| | (oz/in) | Failure Mode | (oz/in) | Failure Mode |
| CE-10 | 33.7 | Clean peel | 6.3 | Clean peel |
| EX-18 | 43.6 | Clean peel | 25.4 | Clean peel |

The thermally reversible crosslinker showed higher peel adhesion to both substrates, but significantly higher adhesion to the porous, rough surface of the POLY substrate. The peel adhesion may be bolstered by a combination of thermal reversibility of the crosslinks, allowing EX-18 to flow and better wet out the surface and possible covalent reactions of the release isocyanate groups with pendant functionality on the substrates.

All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. A molecule represented by the structure 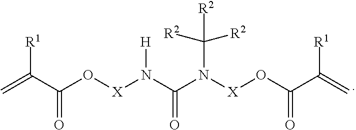
wherein
each R¹ is independently —H or —CH₃,
each X is independently C₂-C₆ alkyl, and
each R² is independently a C₁-C₆ alkyl group or an aromatic group.

2. The molecule of claim 1, wherein R¹ is —H, X is C₂ alkyl, and R² is —CH₃.

3. An article comprising the molecule of claim 1.

4. The article of claim 3, wherein the article comprises a pressure-sensitive adhesive.

5. A curable composition comprising:
a first monomer;
an initiator; and
a crosslinker represented by the structure 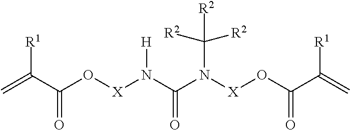
wherein
each R¹ is independently —H or —CH₃,
each X is independently C₂-C₆ alkyl, and
each R² is independently a C₁-C₆ alkyl group or an aromatic group.

6. The curable composition of claim 5, wherein the first monomer comprises a (meth)acrylate monomer.

7. The curable composition of claim 5, wherein the initiator comprises a photoinitiator.

8. The curable composition of claim 5, wherein the crosslinker R¹ is —H, X is C₂ alkyl, and R² is —CH₃.

9. The curable composition of any one of claim 5, wherein the curable composition comprises 0.05 wt. % to 2 wt. % of the crosslinker.

10. The curable composition of claim 5, further comprising a second monomer.

11. The curable composition of claim 5, wherein the composition is essentially free of protic monomers.

12. The curable composition of claim 5, further comprising 0.01 wt. % to 5 wt. % of a chain transfer reagent.

13. The curable composition of claim 5, further comprising up to 60 wt. %, optionally 5 wt. % to 50 wt. % of a tackifier.

14. The curable composition of claim 5, further comprising an additive selected from the group consisting of a pigment, a filler, a plasticizer, and combinations thereof.

15. A pressure-sensitive adhesive comprising the curable composition of claim 5.

16. An article comprising:
a substrate;
the curable composition of claim 5.

17. The article of claim 16, wherein the substrate comprises a poly(ethylene terephthalate) or poly(vinyl chloride) film.

18. The article of claim 16, wherein the article is a double-sided tape.

19. A cured composition prepared from the curable composition of claim 5.

20. A method of preparing an article, the method comprising:
providing a substrate; and
positioning the curable composition of claim 5 adjacent to the substrate.
